(19) United States
(12) Patent Application Publication
(10) Pub. No.: US 2026/0233008 A1
(43) Pub. Date: Aug. 13, 2026

(54) Electrical Circuit for Energy-Efficient Spectral Analysis

(71) Applicant: WISCONSIN ALUMNI RESEARCH FOUNDATION, Madison, WI (US)

(72) Inventors: Tianyu Yang, Madison, WI (US); Kip Ludwig, Middleton, WI (US); Brandon Coventry, Verona, WI (US)

(21) Appl. No.: 19/051,781

(22) Filed: Feb. 12, 2025

(51) Int. Cl. A61N 1/36

(52) U.S. Cl. CPC A61N 1/36139

(57) ABSTRACT

A energy efficient spectral analysis circuit suitable, for example, for implanted biological electronics, sequentially evaluates a Fourier transform of time domain electrical signals using a single multiplier iterating receiving each time domain sample and determining a spectrum over a predetermined, limited number of frequency bins, substantially reducing circuit complexity and power consumption.

### STATEMENT REGARDING FEDERALLY SPONSORED RESEARCH OR DEVELOPMENT

This invention was made with government support under NS129955 awarded by the National Institutes of Health. The government has certain rights in the invention.

### BACKGROUND OF THE INVENTION

The present invention relates generally to electrical circuits for signal analysis and in particular to a circuit providing spectral decomposition with improved energy efficiency.

The spectral analysis of signals finds important use in many applications including, for example, implanted medical devices that rely on limited energy sources such as batteries. In such applications and similar applications where batteries would be difficult to replace, energy-efficient circuitry for spectral analysis would offer considerable advantages. In the medical area this would include reducing invasive surgeries.

Circuitry for spectral analysis frequently implements a version of the fast Fourier transform (FFT). The FFT greatly improves computational efficiency of the Fourier transform by reusing calculations in a way that reduces a the worst-case time of arithmetic operations from O(n²) to O(n log n), where n is the data size of the signal being processed.

### SUMMARY OF THE INVENTION

The present inventors have recognized that circuits implementing the FFT benefit from reducing computational burden; this comes at the cost of reduced energy efficiency both because of the number of complex signal paths and the parallelization of the FFT needed to achieve speed benefits. Accordingly, the present invention performs spectral analysis using a discrete Fourier transform operating serially on a single multiplier and adder element. Although this approach would be expected to be much slower than the FFT, the inventors have determined that high clock rates possible with current integrated circuit technologies allow this approach to work effectively for many important applications while providing important improved energy efficiency. In particular, many neural sensing applications require relatively low sampling rates (e.g., 1 kHz for local field potential recording, 100 Hz for intracranial EEG systems, and minimum 7 kHz for spike detection). At such sampling rates with conventional integrated circuit clock rates, 1600 to 124,000 frequency bins can be processed using the present invention, significantly above minimal requirements for online neural sensing and measurement applications.

More specifically, in one embodiment, the invention provides an electrical circuit for spectral analysis having an input for receiving time domain signal samples; a coefficient memory holding real and imaginary Fourier coefficients; a multiplier; a spectrum memory storing frequency domain signal samples; and control logic. The control logic operates to: (1) sequentially provide each given sample to the multiplier, first, using the multiplier to multiply the sample by the real Fourier coefficient to create a first product and second, using the multiplier to multiply the sample by the second Fourier coefficient to create a second product; (2) use the adder to sum the first and second products for the given sample together for storage in the spectrum memory; and (3) output a spectrum of the time domain signal samples as frequency domain samples obtained from multiple respective values in the spectrum memory.

It is thus a feature of at least one embodiment of the invention to minimize electrical circuit components and related energy consumption through an aggressive serial rather than parallel computational path.

The electrical circuit may further include a frequency bin index holding a frequency bin value and the control logic, for each given sample, sequences through the coefficient memory holding the real and imaginary Fourier coefficients according to a frequency bin value for a predetermined number and range of frequency bin values.

It is thus a feature of at least one embodiment of the invention to allow to constrain the calculation using an a priori knowledge of spectral range and resolution further limiting energy consumption

The electrical circuit may further include an accumulator operating to hold the first product prior to calculation of the second product for each time domain signal sample and to be overwritten for each successive time domain signal sample.

It is thus a feature of at least one embodiment of the invention to minimize the number of multipliers needed through a temporary storage of input values in a compact memory.

The electrical circuit may include a buffer memory positioned between the input and the multiplier.

It is thus a feature of at least one embodiment of the invention to allow asynchronous operation of data acquisition and spectral calculation for reduced energy consumption.

The adder may operate to sum the first and second products to a value in the spectrum memory indexed by the frequency bin value.

It is thus a feature of at least one embodiment of the invention to minimize additional memory and its power consumption.

The electrical circuit may further include a biological sensor for sensing nerve signals and communicating with the input to provide time domain signal samples indicating electrical nerve activity. In some embodiments, the electrical circuit may also include a biological stimulator for providing electrical signals to nerves based on the output spectrum.

It is thus a feature of at least one embodiment of the invention to provide a circuit for spectral decomposition suited to medical and biological applications.

These particular objects and advantages may apply to only some embodiments falling within the claims and thus do not define the scope of the invention.

### BRIEF DESCRIPTION OF THE DRAWINGS

FIG. 1 is a block diagram of an example implementation of the present invention in an implantable medical device providing nerve monitoring and stimulation;

FIG. 2 is a detailed block diagram of a spectrum analysis circuit used in the example of FIG. 1 providing low power consumption; and

FIG. 3 is a flowchart showing operation of the spectrum analysis circuit under control logics.

### DETAILED DESCRIPTION OF THE PREFERRED EMBODIMENT

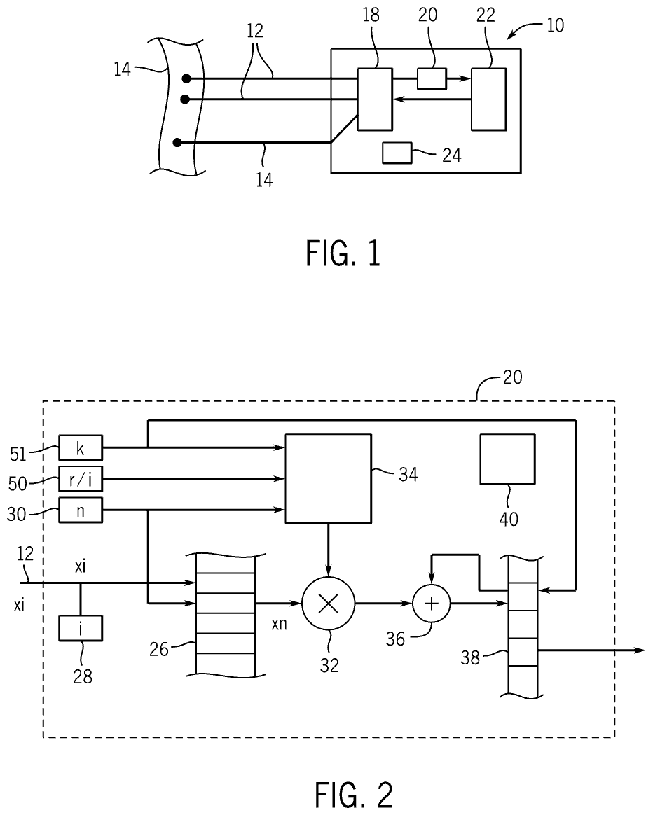

Referring now to FIG. 1, an example embodiment benefiting from the present invention may provide for an implantable electronic device 10, for example, a pacemaker or neurostimulator, to be held within the human body. The implantable electronic device 10 may communicate with electrical sensing leads 12 and with electrical stimulating leads 14 connected to tissue of interest 16, the latter being tissue such as such as a heart, brain or a nerve sheath.

Interface circuitry 18 in the implantable electronic device 10 may digitize the received signals from the tissue of interest 16 via the electrical sensing leads 12, to produce a set of sequential time-domain digital samples xi. In this regard, the interface circuitry 18 may use, for example, an analog to digital converter converting an electrical measurement such as voltage or current to an electronic digital representation, for example, as a 12-bit-wide twos-complement number.

The digital samples xₙ may then be provided to a spectrum analysis circuit 20 operating to extract the frequency spectrum of the signals as will be described in greater detail below.

The frequency spectrum from the spectrum analysis circuit 20 may be provided to an ancillary analysis circuit 22, or transmitted remotely for the purpose of developing control signals for an electrical stimulation signal that will be returned to the interface circuitry 18 to provide stimulating signals on stimulating lead 14 based on the received spectrum.

The circuitry of the implantable electronic device 10 may receive electrical power from power source 24 which will generally be a constrained source of power either being a battery with a finite amount of electrical energy or a wireless power receiver having constrained power transmission capabilities.

Generally, implantable electronic device 10 will be provided with a liquid-impermeable and biocompatible outer housing suitable for implantation. In some instances the housing may be small enough to be injectable, for example, using hypodermic needles, for example, having cross-sectional sides fitting within a 0.05 inch internal diameter hypodermic needle.

In nonlimiting examples, the implantable electronic device 10 may function as an implantable pulse generator (IPG) delivering impulses to the body either to stimulate the heart or brain, for example, as a pacemaker or neurostimulator. An example function for an implantable electronic device 10 is described in in U.S. patent application Ser. No. 18/955,228 entitled: “Localizing Ion-Channel Activation Using Nullifying Waveforms,” filed Nov. 21, 2024, and assigned to the assignee of the present invention and incorporated in full by reference.

The spectrum analysis circuit 20 may operate episodically as triggered by the receipt of a new sample xi, for a predefined number of new samples xi, and the ancillary analysis circuit 22 may operate asynchronously with the spectrum analysis circuit 20, for example, going into a sleep state for periods of time and then waking to interrogate the current spectrum from the spectrum analysis circuit 20 as desired for reduced energy consumption and/or reduced power demand.

Referring now to FIG. 2, a given input stream of data over electrical sensing leads 12, after digitization by the interface circuitry 18, may be received by the spectrum analysis circuit 20 into a buffer memory 26, for example, implemented as a circular buffer. The particular location of each received time-domain digital sample xi in the buffer memory 26 may be determined by a counter value i from a buffer counter 28 incrementing through the locations in the buffer memory 26 with each arriving sample xi.

A second counter value n from a processing counter 30 indexes through the buffer memory 26 asynchronously with the buffer counter 28 to extract a previously stored sample xₙ for forwarding to a multiplier 32 as a first multiplicand. The multiplier 32 also receives a Fourier coefficient from a coefficient memory 34, the latter of which may be a read-only memory indexed by a combination of the counter value n, a frequency bin value k, and a real/imaginary coefficient identifier value r/i, the latter of which is generally a binary number designating either a real or imaginary coefficient. The coefficient memory 34 may be envisioned as a three-dimensional array of data values related to each of the index values n, k, and r/i. These coefficients may be precomputed as follows:
for r/i equals 0 (real), the coefficient is:

$$cos ( 2 π \frac{k n}{N} )$$

for r/i equals 1 (imaginary), the coefficient is

$$- j sin ( 2 π \frac{k n}{N} )$$

where:
k is a frequency bin value,
n is a sequence number of a time domain signal sample, and
N is the total number of samples being analyzed.
j is the complex unit=\sqrt{−1}

Thus as well as being precomputed, the coefficients may be limited by a predetermined known number of bins Nk desired for a particular application and the coefficients for each bin k may be truncated to one quarter cycle of the associated trigonometric function for that value k for space savings, the coefficients being accessed by a simple function of the index variable n determined from the value of k. Generally, the number of frequency bins K and the frame size N may change during runtime to further improve energy efficiency by limiting these values to the minimum necessary. In addition, the clock rate may may be set in run-time according to an expected band limiting of the signal.

The resulting product of the current sample xₙ and it's matching coefficient produced by the multiplier 32 is then provided to an adder/accumulator 36 which adds the current product to a pre-existing accumulated value in an entry of the spectral memory 38 according to an index value of the bin number k. In this respect, the adder/accumulator 36 accumulates products associated with a particular bin number k in a particular memory location which will form a frequency domain sample in the resulting spectrum.

More specifically, a spectrum may be extracted from the spectral memory 38 typically formed of a sequence of values of entries in the spectral memory 38 associated with different bins designated by k. Typically a spectrum will be extracted after a given number of samples xₙ have been analyzed. After such outputting, the values of the spectral memory 38 may be zeroed to calculate the next spectrum.

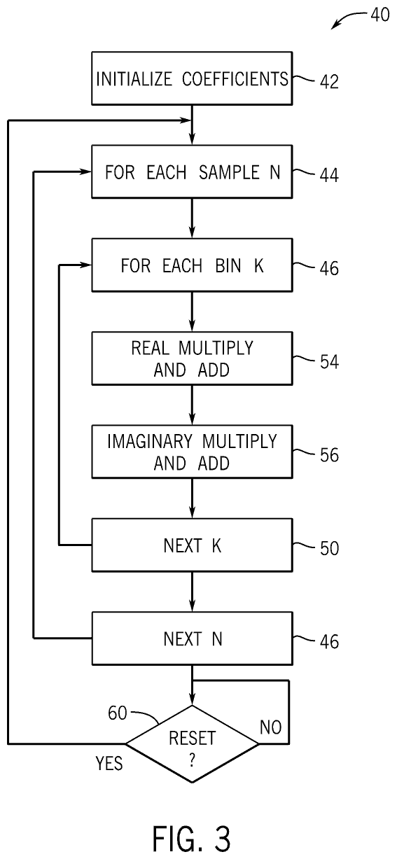

Referring now also to FIGS. 3, the above-described components will be coordinated during operation by control logic 40, for example, implementing a finite state machine. In this regard, the control logic 40 may operate clock rates in excess of 30 MHz, where a clock cycle represents the time required for an arithmetic operation such as a multiplication or addition/accumulation. More generally, slower clock rates may be used for the spectrum analysis circuit 20 to minimize energy consumption when the application permits. In this respect, a particular clock rate may be set to a minimum value to resolve a desired number of frequency bins of a spectrum to be delivered at a given calculation rate suitable for the application. The clock rate of the buffer memory 26 operating asynchronously to the spectrum analysis circuit 20 may be different to match the rate of receipt of time domain samples.

TABLE I
Clock Rates for different common procedures

| | Calculate 10% of the | Calculate 50% of the |
|---|---|---|
| | spectrum (based on | spectrum (based on |
| | half the sampling rate) | half the sampling rate) |
| Intracranial EEG | 60 kHz | 320 kHz |
| LFP | 420 kHz | 2240 kHz |
| Spike detection | 6 kHz | 32 kHz |

As indicated by process block 42, the coefficient memory 34 is precomputed for a desired range of bin numbers k (defining a frequency range of the spectrum) and a given increment between successive values of k (determining a frequency resolution of the spectrum). This definition of k allows the operation of the spectrum analysis circuit 20 to be constrained to minimum energy consumption.

The control logic 40 then enters a first loop defined by process blocks 44 and 46 in which values of n are successively incremented by means of sample counter 30 to obtain samples xₙ. Within each loop of the first loop, a second loop is performed per process blocks 48 and 50 incrementing through k to calculate values for a desired number of bins per bin counter 51.

Within this second loop, the multiplier 32 first multiplies a given sample xₙ by its real coefficient (identified by r/i toggled by counter 52 per process block 54). The output product of the multiplier 32 is then accumulated in a given memory location of the spectral memory 38 as dictated by k. This accumulation makes use of adder/accumulator 36 which sums the current product with the pre-existing value in the spectral memory 38.

As indicated by process block 56, the same multiplier 32 then multiplies the previous sample xₙ by its imaginary coefficient indexed by the toggling of the r/i counter 52. This second product is again added to the given memory location of the spectral memory 38 dictated by k.

Process blocks 54 and 56 are repeated for each of the bins k per counter 51ₙ accumulating all of these values in the memory locations associated with bin k.

Upon conclusion of the analysis of the desired number of bins k this process is repeated with a next sample xₙ, and accumulated values placed in each of the bins k according to the bin counter 51.

Once the defined range of k and n has been traversed, the control logic 40 at process block 60 waits for a reset signal indicating a new spectrum to be calculated and this process is repeated. This reset signal may be produced periodically and may be inhibited during a time when a spectrum is being read out from the spectral memory 38.

By means of the sequential use of a single multiplier 32 and adder/accumulator 36 and by limiting the size of the coefficient memory 34 (according to the resolution of the spectrum and using the coefficient compression noted above), buffer memory 26, and spectral memory 38 according to the desired frequency range and resolution of the spectrum and number of samples and sampling rate of the samples to be analyzed, the required circuitry can be made extremely compact with a corresponding reduction in energy usage.

The implantable electronic device 10 may be readily fabricated using standard integrated circuit techniques, for example, as implemented on field-programmable gate arrays (FPGAs), programmable logic devices (PLDs), programmable systems on chip (PSoC), and such, or in specialized integrated circuits such as constructed in application-specific integrated circuits (ASIC). It will be appreciated that the minimized circuit complexity and integrated circuit area of the present invention allows extremely low energy usage. For example, operating at an 80 MHz clock with 12 bit wide input values and eight frequency bins, the total power consumption will be less than 2 nano Watt-hours or less than 0.15 nanoWatt-hours to process a single time-domain sample. The dynamic power, that is, the difference between power consumption when the circuit is processing a time domain sample versus in a standby state where no processing is active, will be less than 0.5 nano Watt-hours or less than 0.2 nano Watt-hours per a single time-domain sample. These energy consumption values will drop significantly as the clock speed decreases. In this regard, it is anticipated that the clock speed may be reduced to less than 100 kHz, or less than 50 kHz for additional power savings. Importantly, the present invention, in contrast to conventional processors, including von Neumann type processors, need not include energy consuming structures such as a full arithmetic logic unit, random access memory more than four times the time-domain sample size and number of frequency bins, standard computer registers, pipelines, speculative execution, caches or cache management.

It will be appreciated that the present invention may calculate both the Fourier transform and the inverse Fourier transform using the same mechanism described above based on the similarities of these calculations.

While the present invention has been described with respect to an implanted medical device, it will be appreciated that provides a value in a variety of applications where low power consumption is an overriding concern. It will be understood for minimum power consumption, only a single multiplier 32 and single adder/accumulator 36 will be used, the multiplier typically representing a disproportionate energy consumption.

It will be appreciated generally that the buffer memory 26 and spectral memory 38 may be as little as a single storage location with data rapidly moved off of the spectrum analysis circuit 20, for example, by telemetry to further reduce circuitry area and power usage.

Certain terminology is used herein for purposes of reference only and thus is not intended to be limiting. For example, terms such as “upper”, “lower”, “above”, and “below” refer to directions in the drawings to which reference is made. Terms such as “front”, “back”, “rear”, “bottom” and “side”, describe the orientation of portions of the component within a consistent but arbitrary frame of reference which is made clear by reference to the text and the associated drawings describing the component under discussion. Such terminology may include the words specifically mentioned above, derivatives thereof, and words of similar import. Similarly, the terms “first”, “second” and other such numerical terms referring to structures do not imply a sequence or order unless clearly indicated by the context.

When introducing elements or features of the present disclosure and the exemplary embodiments, the articles “a”, “an”, “the” and “said” are intended to mean that there are one or more of such elements or features. The terms “comprising”, “including” and “having” are intended to be inclusive and mean that there may be additional elements or features other than those specifically noted. It is further to be understood that the method steps, processes, and operations described herein are not to be construed as necessarily requiring their performance in the particular order discussed or illustrated, unless specifically identified as an order of performance. It is also to be understood that additional or alternative steps may be employed.

It is specifically intended that the present invention not be limited to the embodiments and illustrations contained herein and the claims should be understood to include modified forms of those embodiments including portions of the embodiments and combinations of elements of different embodiments as come within the scope of the following claims. All of the publications described herein, including patents and non-patent publications, are hereby incorporated herein by reference in their entireties

To aid the Patent Office and any readers of any patent issued on this application in interpreting the claims appended hereto, applicants wish to note that they do not intend any of the appended claims or claim elements to invoke 35 U.S.C. 112 (f) unless the words “means for” or “step for” are explicitly used in the particular claim.

What we claim is:

1. An electrical circuit for spectral analysis comprising:
an input for receiving time domain signal samples;
a coefficient memory holding real and imaginary Fourier coefficients;
a multiplier;
an adder;
a spectrum memory storing frequency domain signal samples; and
control logic operating to:
(1) sequentially provide each given sample to the multiplier, first using the multiplier to multiply the given sample by the real Fourier coefficient to create a first product, and second using the multiplier to multiply the given sample by the second Fourier coefficient to create a second product;
(2) use the adder to add a sum of the first and second products for the given sample in the spectrum memory; and
(3) output a spectrum of the time domain signal samples as frequency domain samples obtained from respective multiple values in the spectrum memory.

2. The electrical circuit of claim 1 further including a frequency bin index holding a frequency bin value and wherein the control logic, for each given sample, sequences through the coefficient memory holding the real and imaginary Fourier coefficients according to a frequency bin value for a predetermined number and range of frequency bin values.

3. The electrical circuit of claim 2 further including an accumulator operating to hold the first product prior to calculation of the second product for each time domain signal sample and to be overwritten for each successive time domain signal sample.

4. The electrical circuit of claim 2 wherein the adder sums the first and second products to a value in the spectrum memory indexed by the frequency bin value.

5. The electrical circuit of claim 1 further including a buffer memory positioned between the input and the multiplier.

6. The electrical circuit of claim 1 wherein the real and imaginary coefficients are of the form of
$$cos ( 2 π \frac{k n}{N} ) ;$$
$$and$$
$$sin ( 2 π \frac{k n}{N} ) ;$$
respectively where:
k is a frequency bin value,
n is a sequence number of a time domain signal sample, and
N is a total number of samples being analyzed.

7. The electrical circuit of claim 1 further including a biological sensor for sensing nerve signals and communicating with the input to provide time domain signal samples indicating electrical nerve activity.

8. The electrical circuit of claim 7 further including a biological stimulator for providing electrical signals to nerves based on the spectrum that is output.

9. The electrical circuit of claim 7 wherein including an electrical battery providing electrical power for operation of the electrical circuit.

10. The electrical circuit of claim 1 wherein the electrical circuit is an integrated circuit.
